(19) United States
(12) Patent Application Publication
(10) Pub. No.: US 2026/0325424 A1
(43) Pub. Date: Oct. 22, 2026

(54) COSMETIC COMPOSITION

(71) Applicants: Murray David Polkinghorne, London (GB); Sakina Mehenni, London (GB)

(72) Inventors: Murray David Polkinghorne, London (GB); Sakina Mehenni, London (GB)

(21) Appl. No.: 19/486,398

(22) PCT Filed: Jun. 28, 2024
(86) PCT No.: PCT/GB2024/051678

§ 371 Date: Nov. 21, 2025

(30) Foreign Application Priority Data
Jun. 29, 2023 (GB) 2309893.2

(51) Int. Cl. A61K 8/49; A61K 8/35; A61Q 19/02; A61Q 19/08

(52) U.S. Cl. CPC A61K 8/498; A61K 8/35; A61Q 19/02; A61Q 19/08

(57) ABSTRACT

The invention provides for a cosmetic composition comprising at least one curcuminoid and quercetin in a phytosomal complex with phosphatidylcholine, together with an aqueous phase comprising at least 10 wt. % rose water; compositions and cosmetic uses are disclosed. The formulation provides synergistic effects to improve the appearance of the skin without therapeutic intent.

### TECHNICAL FIELD OF THE INVENTION

The present invention relates to a cosmetic composition and the use of the cosmetic composition for improving aging attributes.

### BACKGROUND TO THE INVENTION

Skin forms the barrier between the outside world and the human body and as a person ages, the skin ages. Skin can age through a number of mechanisms including oxidative stress, cellular senescence, and DNA damage for example by ultraviolet radiation or pollution.

The physical attributes of aging skin may be defined as skin firmness, skin surface consistency, skin glow and skin tone consistency. Skin surface consistency includes the appearance of pores, wrinkles, fine lines and deep wrinkles. Skin glow refers to the overall appearance of the skin including descriptors such as radiance, brightness, vibrancy, and complexion. Deterioration of these attributes may lead to visibly aged skin. It would

therefore be advantageous to provide a cosmetic composition which may slow, prevent or reverse negative aging skin attributes with improved efficacy. It would be advantageous to provide a cosmetic composition which may slow, prevent or reverse negative aging skin attributes with improved efficacy.

Cosmetics such as face creams, gels or lotions may be used to reduce or prevent the physical attributes of aging skin. Known cosmetics typically comprise a large number of active ingredients which can lead to expensive products with complex manufacturing methods. It would therefore be advantageous to provide a cosmetic composition which comprises fewer ingredients such that it is easier to manufacture and is cheaper to produce. It would be advantageous to provide a cosmetic composition which is easier to manufacture and results in no loss of anti-aging performance efficacy or preferably improved efficacy.

It would furthermore be advantageous to mitigate or overcome at least one problem of the prior art, whether disclosed herein or not.

### SUMMARY OF THE INVENTION

According to a first aspect of the invention there is provided a cosmetic composition comprising: (i) between 0.5 wt. % and 5 wt. % curcumin or curcuminoid and (ii) between 0.5 wt. % and 5 wt. % quercetin or quercetin provided in a phytosomal complex with phosphatidylcholine (sunflower or soy derived lecithin) and (iii) a cosmetically acceptable carrier including an aqueous phase comprising 10 wt. % rose water (hydrosol).

The cosmetic composition of the first aspect of the invention may be advantageous because it may reverse or slow the physical signs of skin aging of the user. The cosmetic composition may be advantageous because it may reverse or reduce the appearance of wrinkles, fine lines, deep wrinkles, pigmentation and/or scarring thereby creating the appearance of more youthful skin. The cosmetic composition may be advantageous because it may improve the firmness, radiance and/or complexion of the skin thereby creating the appearance of more youthful skin.

It has surprisingly been found that combining curcumin and quercetin in the claimed amounts, in a cosmetic composition, provides significant anti-aging efficacy, compared to compositions which do not include the two ingredients in the claimed ranges. In particular, it has been surprisingly found that a combination of the two ingredients in the claimed ranges provides more than an additive effect compared to the individual ingredients and provides a superior effect compared to lower quantities of either ingredient, or lower quantities of the combined ingredients.

The cosmetic composition may modulate cellular senescence markers in the skin for cosmetic purposes without therapeutic effect. A cosmetic composition that modulates skin cellular senescence markers may be advantageous because senescent cells (cells that are damaged or “aged” but do not undergo cell death) have especially deleterious effects on the tissues in which they reside; therefore, efforts to promote autophagy and the apoptotic clearance of these senescent cells through senolytic compositions have the potential to drastically improve skin quality and skin appearance and therefore slow, prevent or reverse negative aging skin attributes. The cosmetic composition that modulates skin cellular senescence markers comprising between 0.5 wt. % and 5 wt. % curcumin may be advantageous because curcumin has been shown to have senolytic properties at these concentrations which lead to an improved aging skin appearance. The cosmetic composition that modulates skin cellular senescence markers comprising between 0.5 wt. % and 5 wt. % quercetin may be advantageous because quercetin has been shown to have senolytic properties at these concentrations which lead to an improved aging skin appearance.

The cosmetic composition may comprise between 0.5 and 5 wt. % curcumin and between 0.5 and 4 wt. % quercetin. The cosmetic composition may comprise between 0.5 and 5 wt. % curcumin and 0.5 and 3 wt. %, 0.5 and 2.5 wt. %, 0.5 and 2 wt. %, 0.5 and 1.5 wt. %, or between 0.5 and 1 wt. % quercetin.

The cosmetic composition may comprise between 0.5 and 4 wt. % curcumin and between 0.5 and 5 wt. % quercetin. The cosmetic composition may comprise between 0.5 and 4 wt. % curcumin and between 0.5 and 4 wt. %, 0.5 and 3 wt. %, 0.5 and 2.5 wt. %, 0.5 and 2 wt. %, 0.5 and 1.5 wt. %, or between 0.5 and 1 wt. % quercetin.

The cosmetic composition may comprise between 0.5 and 3 wt. % curcumin and between 0.5 and 5 wt. % quercetin. The cosmetic composition may comprise between 0.5 and 3 wt. % curcumin and between 0.5 and 4 wt. %, 0.5 and 3 wt. %, 0.5 and 2.5 wt. %, 0.5 and 2 wt. %, 0.5 and 1.5 wt. %, or between 0.5 and 1 wt. % quercetin.

The cosmetic composition may comprise between 0.5 and 2 wt. % curcumin and between 0.5 and 5 wt. % quercetin. The cosmetic composition may comprise between 0.5 and 2 wt. % curcumin and between 0.5 and 4 wt. %, 0.5 and 3 wt. %, 0.5 and 2.5 wt. %, 0.5 and 2 wt. %, 0.5 and 1.5 wt. %, or between 0.5 and 1 wt. % quercetin.

The cosmetic composition may comprise between 0.5 and 1 wt. % curcumin and between 0.5 and 5 wt. % quercetin. The cosmetic composition may comprise between 0.5 and 1 wt. % curcumin and between 0.5 and 4 wt. %, 0.5 and 3 wt. %, 0.5 and 2.5 wt. %, 0.5 and 2 wt. %, 0.5 and 1.5 wt. %, or between 0.5 and 1 wt. % quercetin.

In preferred embodiments the cosmetic composition may comprise between 0.5 and 2 wt. % curcumin and between 0.5 and 2 wt. % quercetin. In further preferred embodiments the cosmetic composition may comprise between 0.5 and 1.5 wt. % curcumin and between 0.5 and 1.5 wt. % quercetin.

According to a second aspect of the invention there is provided a cosmetic composition comprising at least 0.01 wt. % curcumin and at least 0.01 wt. % quercetin.

The curcumin may comprise at least one curcuminoid. The at least one curcuminoid may be as defined or described hereinafter for the third aspect of the invention.

The quercetin may comprise quercetin phytosome. The quercetin phytosome may be as defined or described hereinafter for the third aspect of the invention.

A cosmetic composition can be defined as a composition intended to restore or improve a person's appearance and includes skin care compositions.

The cosmetic composition of the second aspect of the invention may be advantageous because it may reverse or slow the physical signs of skin aging of the user. The cosmetic composition may be advantageous because it may reverse or reduce the appearance of wrinkles, fine lines, deep wrinkles, pigmentation and/or scarring thereby creating the appearance of more youthful skin. The cosmetic composition may be advantageous because it may improve the firmness, radiance and/or complexion of the skin thereby creating the appearance of more youthful skin.

The cosmetic composition may modulate cellular senescence markers in the skin for cosmetic purposes without therapeutic effect. A cosmetic composition that modulates skin cellular senescence markers may be advantageous because senescent cells (cells that are damaged or “aged” but do not undergo cell death) have especially deleterious effects on the tissues in which they reside; therefore, efforts to promote autophagy and the apoptotic clearance of these senescent cells through senolytic compositions have the potential to drastically improve skin quality and skin appearance and therefore slow, prevent or reverse negative aging skin attributes. The cosmetic composition that modulates skin cellular senescence markers comprising at least 0.01 wt. % curcumin may be advantageous because curcumin has been shown to have senolytic properties at these concentrations which lead to an improved aging skin appearance. The cosmetic composition that modulates skin cellular senescence markers comprising at least 0.01 wt. % quercetin may be advantageous because quercetin has been shown to have senolytic properties at these concentrations which lead to an improved aging skin appearance.

The cosmetic composition may comprise between 0.01 and 10 wt. % curcumin or between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % curcumin.

The cosmetic composition may comprise between 0.01 and 10 wt. % quercetin or between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 10 wt. % curcumin and between 0.01 and 10 wt. % quercetin. The cosmetic composition may comprise between 0.01 and 10 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 9 wt. % curcumin and between 0.01 and 10 wt. % quercetin. The cosmetic composition may comprise between 0.01 and 9 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 8 wt. % curcumin and between 0.01 and 10 wt. % quercetin. The cosmetic composition may comprise between 0.01 and 8 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 7 wt. % curcumin and between 0.01 and 10 wt. % quercetin. The cosmetic composition may comprise between 0.01 and 7 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 6 wt. % curcumin and between 0.01 and 10 wt. % quercetin. The cosmetic composition may comprise between 0.01 and 6 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 5 wt. % curcumin and between 0.01 and 10 wt. % quercetin. The cosmetic composition may comprise between 0.01 and 5 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.05 and 5 wt. % curcumin and between 0.01 and 10 wt. % quercetin. The cosmetic composition may comprise between 0.05 and 5 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.1 and 5 wt. % curcumin and between 0.01 and 10 wt. % quercetin. The cosmetic composition may comprise between 0.1 and 5 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.5 and 5 wt. % curcumin and between 0.01 and 10 wt. % quercetin. The cosmetic composition may comprise between 0.5 and 5 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 4 wt. % curcumin and between 0.01 and 10 wt. % quercetin. The cosmetic composition may comprise between 0.01 and 4 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.1 and 7 wt. % curcumin and between 0.01 and 10 wt. % quercetin. The cosmetic composition may comprise between 0.1 and 4 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.5 and 4 wt. % curcumin and between 0.01 and 10 wt. % quercetin. The cosmetic composition may comprise between 0.5 and 4 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 3 wt. % curcumin and between 0.01 and 10 wt. % quercetin. The cosmetic composition may comprise between 0.01 and 3 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, n0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.1 and 8 wt. % curcumin and between 0.01 and 10 wt. % quercetin. The cosmetic composition may comprise between 0.1 and 3 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.5 and 3 wt. % curcumin and between 0.01 and 10 wt. % quercetin. The cosmetic composition may comprise between 0.5 and 3 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 2 wt. % curcumin and between 0.01 and 10 wt. % quercetin. The cosmetic composition may comprise between 0.01 and 2 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.05 and 9 wt. % curcumin and between 0.01 and 10 wt. % quercetin. The cosmetic composition may comprise between 0.05 and 9 wt. % curcumin and between 0.01 and 9 wt. %, 0.01 and 8 wt. %, wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.1 and 2 wt. % curcumin and between 0.01 and 10 wt. % quercetin. The cosmetic composition may comprise between 0.1 and 2 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.5 and 2 wt. % curcumin and between 0.01 and 10 wt. % quercetin. The cosmetic composition may comprise between 0.5 and 2 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.01 and 9 wt. % curcumin and between 0.01 and 10 wt. % quercetin. The cosmetic composition may comprise between 0.01 and 9 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.05 and 1 wt. % curcumin and between 0.01 and 10 wt. % quercetin. The cosmetic composition may comprise between 0.05 and 1 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.1 and 1 wt. % curcumin and between 0.01 and 10 wt. % quercetin. The cosmetic composition may comprise between 0.1 and 1 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

The cosmetic composition may comprise between 0.5 and 1 wt. % curcumin and between 0.01 and 10 wt. % quercetin. The cosmetic composition may comprise between 0.5 and 1 wt. % curcumin and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin.

In preferred embodiments the cosmetic composition may comprise between 0.01 and 7 wt. % curcumin and between 0.01 and 7 wt. % quercetin. In further preferred embodiments the cosmetic composition may comprise between 0.1 and 5 wt. % curcumin and between 0.1 and 5 wt. % quercetin. In further preferred embodiments the cosmetic composition may comprise between 0.1 and 3 wt. % curcumin and between 0.1 and 3 wt. % quercetin. In further preferred embodiments the cosmetic composition may comprise between 0.5 and 2 wt. % curcumin and between 0.5 and 2 wt. % quercetin. In further preferred embodiments the cosmetic composition may comprise between 0.5 and 1.5 wt. % curcumin and between 0.5 and 1.5 wt. % quercetin.

In preferred embodiments the cosmetic composition may comprise approximately 1 wt. % curcumin and approximately 1 wt. % quercetin. This embodiment may be advantageous because it may reverse or reduce the appearance of wrinkles, fine lines, deep wrinkles and/or pigmentation and/or this may improve the firmness, radiance and/or complexion of the skin thereby creating the appearance of more youthful skin.

The ratio of the amount of curcumin to the amount of quercetin in the cosmetic composition of the first aspect of the invention or of the second aspect of the invention may be between 10:1 and 1:10. The ratio of the amount of curcumin to the amount of quercetin in the cosmetic composition of the first aspect of the invention or of the second aspect of the invention may be between 9:1 and 1:10, 8:1 and 1:10, 7:1 and 1:10, 6:1 and 1:10, 5:1 and 1:10, 4:1 and 1:10, 3:1 and 1:10 or between 2:1 and 1:10. The ratio of the amount of curcumin to the amount of quercetin in the cosmetic composition of the first aspect of the invention or of the second aspect of the invention may be between 10:1 and 1:9, 10:1 and 1:8, 10:1 and 1:7, 10:1 and 1:6, 10:1 and 1:5, 10:1 and 1:4, 10:1 and 1:3 or between 10:1 and 1:2. The ratio of the amount of curcumin to the amount of quercetin in the cosmetic composition of the first aspect of the invention or of the second aspect of the invention may be between 9:1 and 1:9, 8:1 and 1:8, 7:1 and 1:7, 6:1 and 1:6, 5:1 and 1:5, 4:1 and 1:4, 3:1 and 1:3 or between 2:1 and 1:2.

The ratio of the amount of curcumin to the amount of quercetin in the cosmetic composition of the first aspect of the invention or of the second aspect of the invention maybe between 1.9:1 and 1:1.9 or between 1.8:1 and 1:1.8, 1.7:1 and 1:1.7, 1.6:1 and 1:1.6, 1.5:1 and 1:1.5, 1.4:1 and 1:1.4, 1.3:1 and 1:1.3, 1.2:1 and 1:1.2 or between 1.1:1 and 1:1.1. In preferred embodiments the ratio of curcumin to quercetin in the cosmetic composition of the first aspect of the invention or of the second aspect of the invention may be approximately 1:1. This embodiment may be advantageous because it may reverse or reduce the appearance of wrinkles, fine lines, deep wrinkles and/or pigmentation and/or this may improve the firmness, radiance and/or complexion of the skin thereby creating the appearance of more youthful skin.

According to a third aspect of the invention there is provided a topical cosmetic composition comprising at least 0.01 wt. % of at least one curcuminoid and at least 0.01 wt. % quercetin phytosome.

The curcuminoid may comprise curcumin or a metabolite of curcumin. The curcuminoid may be selected from the group consisting of tetrahydrocurcuminoid (THC), hexahydrocurcuminoid (HHC) and octahydrocurcuminoid (OHC), or any combination thereof.

The at least one curcuminoid may comprise tetrahydrocurcuminoid (THC), hexahydrocurcuminoid (HHC) and octahydrocurcuminoid (OHC).

The at least one curcuminoid may be a mixture of curcuminoids wherein the mixture may comprise at least 2 wt. % THC. The mixture may comprise at least 2.5 wt. % or at least 3 wt. % THC. The mixture may comprise no more than 8 wt. % or no more than 7 wt. %, 6 wt. % or no more than 5 wt. % THC. The mixture may comprise between 2.5 and 8 wt. % THC or between 3 and 6 wt. % or between 3 and 5 wt. % THC.

The at least one curcuminoid be a mixture of curcuminoids wherein the mixture may comprise at least 10 wt. % HHC. The mixture may comprise at least 11 wt. %, 12 wt. %, 13 wt. % or at least 15 wt. % HHC. The mixture may comprise no more than 25 wt. % or no more than 24 wt. %, 23 wt. %, 22 wt. %, 21 wt. % or no more than 20 wt. % HHC. The mixture may comprise between 10 and 25 wt. % THC or between 11 and 24 wt. %, 12 and 23 wt. %, 13 and 22 wt. %, 14 and 22 wt. %, or between 15 and 20 wt. % THC.

The at least one curcuminoid be a mixture of curcuminoids wherein the mixture may comprise at least 60 wt. % OHC. The mixture may comprise at least 65 wt. %, 70 wt. % or at least 75 wt. %. The mixture may comprise no more than 90 wt. % or no more than 85 wt. %, or no more than 80 wt. % OHC. The mixture may comprise between 60 and 90 wt. % OHC or between 65 and 85 wt. %, 70 and 80 wt. % or between 75 and 80 wt. % THC.

The at least one curcuminoid may be a mixture of curcuminoids wherein the mixture may comprise between 75 and 80 wt. % of tetrahydrocurcuminoids (THC), between 15 and 20 wt. % of hexahydrocurcuminoids (HHC) and between 3 and 5 wt. % of octahydrocurcuminoids (OHC). “Quercetin phytosome” comprises a non-covalent complex of quercetin with at least one phospholipid (e.g. phosphatidylcholine (PC) derived from sunflower or soy lecithin) prepared by solvent evaporation or anti-solvent precipitation. The term “quercetin equivalent” expresses the mass of quercetin present in the complex (i.e., excluding PC). A topical cosmetic composition comprising quercetin phytosome may be advantageous because it may have improved absorption through the skin barrier.

The topical cosmetic composition may comprise between 0.01 and 10 wt. % of at least one curcuminoid or between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % of at least one curcuminoid.

The topical cosmetic composition may comprise between 0.01 and 10 wt. % quercetin phytosome or between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 10 wt. % of at least one curcuminoid and between 0.01 and 10 wt. % quercetin phytosome. The topical cosmetic composition may comprise between 0.01 and 10 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 9 wt. % of at least one curcuminoid and between 0.01 and 10 wt. % quercetin phytosome. The topical cosmetic composition may comprise between 0.01 and 9 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 8 wt. % of at least one curcuminoid and between 0.01 and 10 wt. % quercetin phytosome. The topical cosmetic composition may comprise between 0.01 and 8 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 7 wt. % of at least one curcuminoid and between 0.01 and 10 wt. % quercetin phytosome. The topical cosmetic composition may comprise between 0.01 and 7 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 6 wt. % of at least one curcuminoid and between 0.01 and 10 wt. % quercetin phytosome. The topical cosmetic composition may comprise between 0.01 and 6 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 5 wt. % of at least one curcuminoid and between 0.01 and 10 wt. % quercetin phytosome. The topical cosmetic composition may comprise between 0.01 and 5 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.05 and 5 wt. % of at least one curcuminoid and between 0.01 and 10 wt. % quercetin phytosome. The topical cosmetic composition may comprise between 0.05 and 5 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.1 and 5 wt. % of at least one curcuminoid and between 0.01 and 10 wt. % quercetin phytosome. The topical cosmetic composition may comprise between 0.1 and 5 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.5 and 5 wt. % of at least one curcuminoid and between 0.01 and 10 wt. % quercetin phytosome. The topical cosmetic composition may comprise between 0.5 and 5 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 4 wt. % of at least one curcuminoid and between 0.01 and 10 wt. % quercetin phytosome. The topical cosmetic composition may comprise between 0.01 and 4 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.1 and 4 wt. % of at least one curcuminoid and between 0.01 and 10 wt. % quercetin phytosome. The topical cosmetic composition may comprise between 0.1 and 4 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.5 and 4 wt. % of at least one curcuminoid and between 0.01 and 10 wt. % quercetin phytosome. The topical cosmetic composition may comprise between 0.5 and 4 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 3 wt. % of at least one curcuminoid and between 0.01 and 10 wt. % quercetin phytosome. The topical cosmetic composition may comprise between 0.01 and 3 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.1 and 3 wt. % of at least one curcuminoid and between 0.01 and 10 wt. % quercetin phytosome. The topical cosmetic composition may comprise between 0.1 and 3 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.5 and 3 wt. % of at least one curcuminoid and between 0.01 and 10 wt. % quercetin phytosome. The topical cosmetic composition may comprise between 0.5 and 3 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 2 wt. % of at least one curcuminoid and between 0.01 and 10 wt. % quercetin phytosome. The topical cosmetic composition may comprise between 0.01 and 2 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.05 and 2 wt. % of at least one curcuminoid and between 0.01 and 10 wt. % quercetin phytosome. The topical cosmetic composition may comprise between 0.05 and 2 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.18 and 18 wt. % of at least one curcuminoid and between 0.01 and 10 wt. % quercetin phytosome. The topical cosmetic composition may comprise between 0.1 and 2 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.5 and 2 wt. % of at least one curcuminoid and between 0.01 and 10 wt. % quercetin phytosome. The topical cosmetic composition may comprise between 0.5 and 2 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.01 and 1 wt. % of at least one curcuminoid and between 0.01 and 10 wt. % quercetin phytosome. The topical cosmetic composition may comprise between 0.01 and 1 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.05 and 19 wt. % of at least one curcuminoid and between 0.01 and 10 wt. % quercetin phytosome. The topical cosmetic composition may comprise between 0.05 and 1 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.1 and 1 wt. % of at least one curcuminoid and between 0.01 and 10 wt. % quercetin phytosome. The topical cosmetic composition may comprise between 0.1 and 1 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

The topical cosmetic composition may comprise between 0.5 and 1 wt. % of at least one curcuminoid and between 0.01 and 10 wt. % quercetin phytosome. The topical cosmetic composition may comprise between 0.5 and 1 wt. % of at least one curcuminoid and between 0.01 and 9 wt. %, 0.01 and 8 wt. %, 0.01 and 7 wt. %, 0.01 and 6 wt. %, 0.01 and 5 wt. %, 0.05 and 5 wt. %, 0.1 and 5 wt. %, 0.5 and 5 wt. %, 0.01 and 4 wt. %, 0.1 and 4 wt. %, 0.5 and 4 wt. %, 0.01 and 3 wt. %, 0.1 and 3 wt. %, 0.5 and 3 wt. %, 0.01 and 2 wt. %, 0.05 and 2 wt. %, 0.1 and 2 wt. %, 0.5 and 2 wt. %, 0.01 and 1 wt. %, 0.05 and 1 wt. %, 0.1 and 1 wt. % or between 0.5 and 1 wt. % quercetin phytosome.

In preferred embodiments the topical cosmetic composition may comprise between 0.5 and 2 wt. % of at least one curcuminoid and between 0.5 and 2 wt. % quercetin phytosome. In further preferred embodiments the topical cosmetic composition may comprise between 0.5 and 1.5 wt. % of at least one curcuminoid and between 0.5 and 1.5 wt. % quercetin phytosome.

In preferred embodiments the topical cosmetic composition may comprise approximately 1 wt. % of at least one curcuminoid and approximately 1 wt. % quercetin phytosome. In further preferred embodiments the topical cosmetic composition may comprise approximately 1 wt. % of at least one curcuminoid and approximately 1 wt. % quercetin phytosome wherein the approximately 1 wt. % of at least one curcuminoid may comprise between 75 and 80 wt. % of tetrahydrocurcuminoids (THC), between 15 and 20 wt. % of hexahydrocurcuminoids (HHC) and between 3 and 5 wt. % of octahydrocurcuminoids (OHC). This embodiment may be advantageous because it may reverse or reduce the appearance of wrinkles, fine lines, deep wrinkles and/or pigmentation and/or this may improve the firmness, radiance and/or complexion of the skin thereby creating the appearance of more youthful skin.

The ratio of the amount of the at least one curcuminoid to the amount of quercetin phytosome in the topical cosmetic composition may be between 10:1 and 1:10. The ratio of the amount of the at least one curcuminoid to the amount of quercetin phytosome in the topical cosmetic composition may be between 9:1 and 1:10, 8:1 and 1:10, 7:1 and 1:10, 6:1 and 1:10, 5:1 and 1:10, 4:1 and 1:10, 3:1 and 1:10 or between 2:1 and 1:10. The ratio of the amount of the at least one curcuminoid to the amount of quercetin phytosome in the topical cosmetic composition may be between 10:1 and 1:9, 10:1 and 1:8, 10:1 and 1:7, 10:1 and 1:6, 10:1 and 1:5, 10:1 and 1:4, 10:1 and 1:3 or between 10:1 and 1:2. The ratio of the amount of the at least one curcuminoid to the amount of quercetin phytosome in the topical cosmetic composition may be between 9:1 and 1:9, 8:1 and 1:8, 7:1 and 1:7, 6:1 and 1:6, 5:1 and 1:5, 4:1 and 1:4, 3:1 and 1:3 or between 2:1 and 1:2.

The ratio of the amount of the at least one curcuminoid to the amount of quercetin phytosome in the topical cosmetic composition may be between 1.9:1 and 1:1.9 or between 1.8:1 and 1:1.8, 1.7:1 and 1:1.7, 1.6:1 and 1:1.6, 1.5:1 and 1:1.5, 1.4:1 and 1:1.4, 1.3:1 and 1:1.3, 1.2:1 and 1:1.2 or between 1.1:1 and 1:1.1. In preferred embodiments the ratio of the amount of the at least one curcuminoid to the amount of quercetin phytosome in the topical curcumin may be approximately 1:1. This embodiment may be advantageous because it may reverse or reduce the appearance of wrinkles, fine lines, deep wrinkles and/or pigmentation and/or this may improve the firmness, radiance and/or complexion of the skin thereby creating the appearance of more youthful skin.

The topical cosmetic composition may be referred to as a “cosmetic composition” in the following statements. The following statements may apply to the cosmetic compositions of the first, the second or the third aspects of the invention.

The cosmetic composition of any of the aspects of the invention may comprise a cosmetically acceptable carrier. The amount of acceptable carrier may be no more than 99.8 wt. %. The amount of acceptable carrier may be no more than 99.7 wt. %, 99.6 wt. %, 99.5 wt. %, 99.4 wt. %, 99.3 wt. %, 99.2 wt. %, 99 wt. %, 95 wt. %, 90 wt. %, 85 wt. % or no more than 80 wt. %. The amount of acceptable carrier may be at least 50 wt. %. The amount of acceptable carrier may be at least 60 wt. %, 70 wt. %, 80 wt. %, 90 wt. %, 95 wt. %, 96 wt. %, 97 wt. % or at least 98 wt. %. The amount of acceptable carrier may be between 80 wt. % and 99.8 wt. %. The amount of acceptable carrier may be between 82 wt. % and 99.5 wt. %, 84 wt. % and 99 wt. %, 82 wt. % and 99.5 wt. %, 84 wt. % and 99.5 wt. %, 86 wt. % and 99.5 wt. %, 88 wt. % and 99.5 wt. %, 90 wt. % and 99.5 wt. %, 90 wt. % and 99 wt. %, 92 wt. % and 99 wt. %, 94 wt. % and 99 wt. %, 95 wt. % and 99 wt. %, 96 wt. % and 99 wt. %, 97 wt. % and 99 wt. % or between 97.5 wt. % and 98.5 wt. %.

The cosmetically acceptable carrier may comprise at least one aqueous medium. The aqueous medium may be water. The aqueous medium may be 10 wt. % rose water (hydrosol). Rose water (hydrosol) means the steam distilled Rosa damascena floral water, comprising water-soluble phenolic constituents. The aqueous medium may be a gel. The gel may be selected from the group consisting of: aloe vera gel, orange gel, cucumber gel, watermelon gel, agave, rosehip gel and flax gel and any combination thereof. The amount of the aqueous medium may be between 45 and 90 wt. % of the total weight of the cosmetic composition.

The cosmetically acceptable carrier may comprise at least one oil. The oil may be selected from the group consisting of: mineral oil, oil from animal origin, oil from plant origin, oil from vegetable origin and synthetic oil and any combination thereof. The amount of the oil may be between 3 and 30 wt. % of the total weight of the cosmetic composition.

The mineral oil may be selected from the group consisting of: liquid paraffin, petroleum oil, liquid petroleum, paraffin oil, liquid paraffin, white oil and white mineral oil. The oil from animal origin may be lanolin and any combination thereof. The oil from plant or vegetable origin may be selected from the group consisting of: shea butter, argan oil, avocado oil, castor oil, olive oil, jojoba oil, sesame oil, peanut oil, grapeseed oil, rapeseed oil, coconut oil, hazelnut oil, palm oil, apricot kernel oil, coconut oil, rice bran oil, corn germ oil, wheat germ oil, soybean oil, sunflower oil, primrose oil, safflower oil, and rye oil and any combination thereof.

The synthetic oil may be selected from the group consisting of: butyl myristate, isopropyl myristate, cetyl myristate, isopropyl palmitate, isopropyl adipate, ethylhexyl adipate, butyl stearate, hexadecyl stearate, isopropyl stearate, octyl stearate, isocetyl stearate, decyl oleate, hexyl lauryl propylene, silicone oils, isoparaffins, poly-alpha-olefins and esters derived from lanoic acid such as isopropyl lanolate and isocetyl lanolate and any combination thereof.

The cosmetically acceptable carrier may comprise at least one additional component. The at least one additional component may be selected from a group consisting of: a binder, a thickener or thickening agent, a film former, an oil, a solvent, a colourant, a fragrance, a preservative, an emulsifier, an antioxidant, a vitamin, a UV protecting agent (including those providing an SPF) and a stabiliser and any combination thereof.

The binder may be selected from the group consisting of: butylene glycol, polysorbates, glyceryl stearate, cetearyl alcohol, cetyl alcohol, stearyl alcohol, stearic acid, polyethylene glycol, triethanolamine and palmitates and any combination thereof.

The thickener or thickening agent or the stabiliser may be selected from the group consisting of: polyethylene glycol, polyacrylic acid, hyaluronic acid, stearic acid, xanthan gum, acacia gum, sclerotium gum, petroleum jelly, waxes, fatty alcohols, fatty acid esters, fatty acid amides, clays, silicas, acrylic copolymers, cellulosic polymers (such as hydroxyethyl cellulose, hydroxypropyl cellulose, carboxymethyl cellulose and cationic hydroxypropyl cellulose) and polysaccharides and any combination thereof.

The film former may be a polymer. The film former may be selected from the group consisting of: PVP, acrylate, acrylamide, methacrylate and various copolymers and any combination thereof.

The oil may be selected from the group consisting of: hydrocarbon oil and wax, silicone oil, fatty acid derivative, diglyceride, triglyceride, vegetable oil, vegetable oil derivatives, alkyl or alkenyl ester, lanolin and its derivatives, wax ester, beeswax derivatives and sterol and any combinations thereof. The hydrocarbon oil may be selected from the group consisting of: petrolatum, mineral oil, micro-crystalline wax, polyalkene, paraffins, ceresin, polyethylene, perhydrosqualene, poly alpha-olefin and hydrogenated polyisobutene and any combination thereof. Suitable diglycerides or triglycerides may be selected from the group consisting of: castor oil, soy bean oil, derivatized soybean oils such as maleated soy bean oil, safflower oil, cottonseed oil and derivatized cottonseed oil, corn oil, walnut oil, peanut oil, olive oil, cod liver oil, almond oil, palm oil and sesame oil, sunflower seed oil, coconut oil and derivatized coconut oil, and cocoa butter and any combination thereof.

The solvent may be selected from the group consisting of: ethyl alcohol, isopropanol, acetone, ethylene glycol monoethyl ether, diethylene glycol monobutyl ether, diethylene glycol monoethyl ether, polyalkylene glycol, propylene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol and derivatives and any combination thereof.

The colourant may be a dye or a pigment.

The fragrance may be an essential oil. The fragrance may be an artificial or synthetic fragrance. The fragrance may be selected from the group consisting of: lavender oil, chamomile oil, rose oil, citrus oil, tea tree oil, peppermint oil, bergamot oil, rice flower oil and eucalyptus oil and any combination thereof.

The preservative may be selected from the group consisting of: phenoxyethanol, paraben, potassium sorbate, sodium benzoate, benzyl alcohol, methyl paraben, diazolidinyl urea, benzoic acid, sorbic acid, BHT, triclosan, imidazolidinyl urea, DMDM hydantoin, propylene glycol, dehydroacetic acid, iodopropynyl butylcarbamate, ethylhexylglycerin, 1,2-octanediol, butyl hydroxyanisole, salicylic acid and benzalkonium chloride and any combination thereof.

The emulsifier may be selected from the group consisting of: cetearyl alcohol, glycerol monostearate, polysorbate, glycerol, stearic acid, lecithin, xanthan gum, cetyl alcohol, polyethylene glycol, isopropyl myristate, a glucoside derivative, siloxane and isopropyl palmitate and any combination thereof.

The at least one additional component of the cosmetically acceptable carrier may be an active and/or other ingredient. The at least one additional component may be selected from the group consisting of: hyaluronic acid, retinal, glycolic acid, resveratrol, niacinamide, ubiquinone, squalane, pre- and pro-biotics, algae, seaweed, black pepper and/or black pepper plants, honey, rice and/or rice plants, pomegranates and/or pomegranate seeds, roses and/or rose bushes, rosemary, oregano, olives and/or olive trees, thyme and/or thyme plants, stone pines, calendula, argan trees and/or argan tree kernels, avocados and/or avocado trees, clay, charcoal, coconuts and/or coconut trees, liquorice (Glycyrrhiza glabra) and/or liquorice roots, lavender and/or lavender plants, raspberries and or raspberry seeds, rice bran and/or rice bran plants, rosehip and/or rosehip plants, blueberries and/or blueberry plants, walnuts and/or walnut plants, hazelnuts and/or hazelnut plants, almonds and/or almond plants, Vitellaria paradoxa, Simmondsia chinensis, Camellia sinensis, Tamarix aphylla, Telephium imperati, Ulmus rubra, Cirsium creticum, Lycium shawii, Crataegus azarolus, Centaurea acaulis, Verbascum sinuatum, Quercus faginea, Lupinus micranthus, Phyllanthus niruri, Hypecoum procumbens, Gentiana acaulis, Marrubium vulgare, Artemisia herba-alba, Zingiber officinale, Juniperus phoenicea, Origanum majorana, Eruca sativa, Carum carvi, Matricaria chamomilla, Ziziphus spina-christi, Echinops spinosissimus, Tussilago farfara, Echinops ritro, Carduus nutans, Nicotiana tabacum, Pistacia lentiscus, Carthamus tinctorius, Curcuma longa, Vitis vinifera, rose water (hydrosol), vitamins, minerals, antioxidants and mushrooms such as reishi, tremella, shiitake, cordyceps, coprinus, chaga, and trametes versicolor mushrooms, and any combination thereof.

In preferred embodiments the cosmetic composition of any of the aspects of the invention may comprise rose water (hydrosol). In further preferred embodiments the cosmetic composition of any of the aspects of the invention may comprise at least one component selected from the group consisting of: rose water (hydrosol), grapeseed oil, honey and aloe vera or any combination thereof.

The cosmetic composition of any of the aspects of the invention may comprise at least 5 wt. % rose water (hydrosol). The cosmetic composition may comprise at least 6 wt. %, 7 wt. %, 8 wt. %, 9 wt. % or at least 10 wt. % rose water (hydrosol). The cosmetic composition may comprise no more than 30 wt. % rose water (hydrosol). The cosmetic composition may comprise no more than 25 wt. %, 20 wt. %, 15 wt. % or no more than 12 wt. % rose water (hydrosol). The cosmetic composition may comprise between 5 wt. % and 30 wt. % rose water (hydrosol). The cosmetic composition may comprise between 5 wt. % and 25 wt. %, 5 wt. % and 20 wt. %, 5 wt. % and 15 wt. %, 10 wt. % and 20 wt. %, 10 wt. % and 15 wt. % or between 5 wt. % and 10 wt. % rose water (hydrosol). The cosmetic composition may comprise approximately 10 wt. % rose water (hydrosol).

The cosmetic composition of any of the aspects of the invention may comprise at least 2 wt. % grapeseed oil. The cosmetic composition may comprise at least 3 wt. %, 4 wt. % or at least 5 wt. % grapeseed oil. The cosmetic composition may comprise no more than 15 wt. % grapeseed oil. The cosmetic composition may comprise no more than 12 wt. %, 10 wt. %, 8 wt. % or no more than 7 wt. % grapeseed oil. The cosmetic composition may comprise between 2 wt. % and 15 wt. % grapeseed oil. The cosmetic composition may comprise between 3 wt. % and 12 wt. %, 4 wt. % and 10 wt. %, 5 wt. % and 10 wt. %, 4 wt. % and 8 wt. % or between 4 wt. % and 8 wt. % grapeseed oil. The cosmetic composition may comprise approximately 5 wt. % grapeseed oil.

The cosmetic composition of any of the aspects of the invention may comprise at least 5 wt. % honey. The cosmetic composition may comprise at least 10 wt. %, 15 wt. %, 20 wt. %, 25 wt. % or at least 30 wt. % honey. The cosmetic composition may comprise no more than 50 wt. % honey. The cosmetic composition may comprise no more than 45 wt. %, 40 wt. % or no more than 35 wt. % honey. The cosmetic composition may comprise between 5 wt. % and 50 wt. % honey. The cosmetic composition may comprise between 10 wt. % and 45 wt. %, 15 wt. % and 40 wt. %, 20 wt. % and 40 wt. %, 25 wt. % and 35 wt. %, 25 wt. % and 30 wt. % or between 30 wt. % and 35 wt. % honey. The cosmetic composition may comprise approximately 30 wt. % honey.

The cosmetic composition of any of the aspects of the invention may comprise at least 20 wt. % aloe vera. The cosmetic composition may comprise at least 30 wt. %, 35 wt. %, 40 wt. %, 45 wt. % or at least 50 wt. % aloe vera. The cosmetic composition may comprise no more than 70 wt. % aloe vera. The cosmetic composition may comprise no more than 65 wt. %, 60 wt. % or no more than 55 wt. % aloe vera. The cosmetic composition may comprise between 20 wt. % and 70 wt. % aloe vera. The cosmetic composition may comprise between 30 wt. % and 65 wt. %, 40 wt. % and 60 wt. %, 45 wt. % and 60 wt. %, 45 wt. % and 55 wt. %, 45 wt. % and 50 wt. % or between 50 wt. % and 55 wt. % aloe vera. The cosmetic composition may comprise approximately 50 wt. % aloe vera.

The cosmetic composition of any of the aspects of the invention is preferably a topical cosmetic composition.

The topical cosmetic composition may be a balm. The topical cosmetic composition may be a cream. The topical cosmetic composition may be a lotion. The topical cosmetic composition may be a gel. The topical cosmetic composition may be a mousse. The topical cosmetic composition may be an oil. The topical cosmetic composition may be a serum. The topical cosmetic composition may be a solution. The topical cosmetic composition may be a spray.

According to a fourth aspect of the invention there is provided a method of improving the appearance of skin, hair and/or nails comprising topically applying a cosmetic composition of the first, second or third aspect of the invention to the skin, hair and/or nails.

The amount of the cosmetic composition used for each application may be between 0.5 mL and 10 mL applied per ~800 cm² of skin/hair/nails. The amount of the cosmetic composition used for each application may be between 0.5 mL and 7 mL or between 0.5 mL and 5 mL, 0.5 mL and 4 mL, 0.5 mL and 3 mL or between 0.5 mL and 2 mL applied per ~800 cm² of skin/hair/nails. The amount of the cosmetic composition used for each application may be approximately 1 mL applied per ~800 cm² of skin/hair/nails.

The cosmetic composition may be applied to the face (including under the eyes) and/or neck. The cosmetic composition may be applied to the body. The cosmetic composition may be applied to both the body and the face (including under the eyes) and/or neck. The cosmetic composition may be applied to both the hair and/or nails and the face (including under the eyes) and/or neck. The cosmetic composition may be applied to both the hair and/or nails and the body. The cosmetic composition may be applied to all of the hair and/or nails and the body and the face (including under the eyes) and/or neck.

Improving the appearance of skin may comprise cosmetically improving at least one visible sign of aging. Improving the appearance of skin may comprise reversing at least one visible sign of aging. Improving the appearance of skin may comprise preventing at least one visible sign of aging. The visible sign of aging may be selected from the group consisting of: wrinkles, deep wrinkles, fine lines, derated radiance, reduced firmness, derated pore quality, increased pigmentation and increased scarring and any combination thereof.

According to a fifth aspect of the invention there is provided the cosmetic composition of the first, second or third aspect of the invention for use in improving the appearance of skin.

According to a sixth aspect of the invention there is provided use of a cosmetic composition of the first, second or third aspect of the invention for cosmetically improving the appearance of skin, hair and/or nails.

The following statements apply to the fifth and sixth aspects of the invention. Improving the appearance of skin may comprise cosmetically improving at least one visible sign of aging. Improving the appearance of skin may comprise reversing at least one visible sign of aging. Improving the appearance of skin may comprise preventing at least one visible sign of aging. Improving the appearance of skin may comprise selectively inducing the death of senescent cells of the skin. The visible sign of aging may be selected from the group consisting of: wrinkles, deep wrinkles, fine lines, derated radiance, reduced firmness, derated pore quality, increased pigmentation and increased scarring and any combination thereof.

### DETAILED DESCRIPTION OF THE INVENTION

In order that the invention may be more clearly understood an embodiment thereof will now be described, by way of example only, with reference to the accompanying drawings, of which:

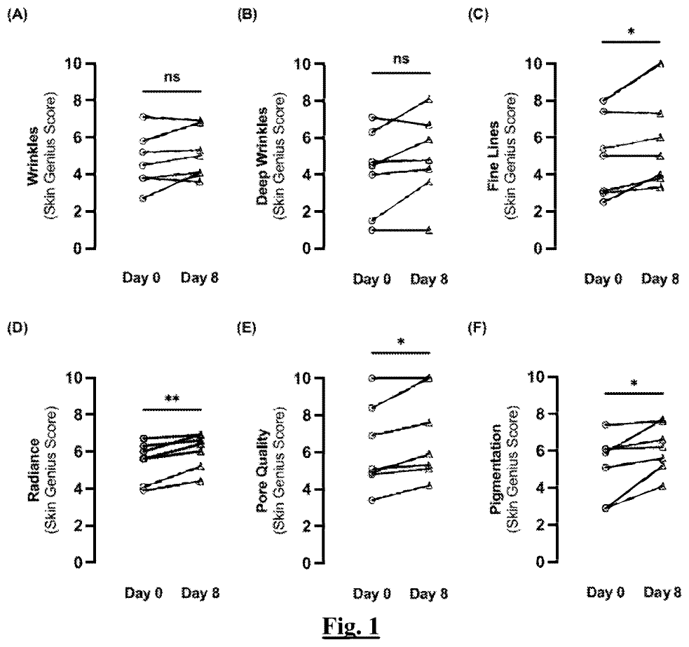

FIG. 1 shows 6 graphs indicating the change of 6 skin attributes after 8 days of topical application of a first embodiment of a cosmetic composition of the first aspect of the invention wherein the cosmetic composition comprises 1 wt. % quercetin and 1 wt. % curcumin and the skin attributes are (A) wrinkles, (B) deep wrinkles, (C) fine lines, (D) radiance, (E) pore quality and (F) pigmentation.

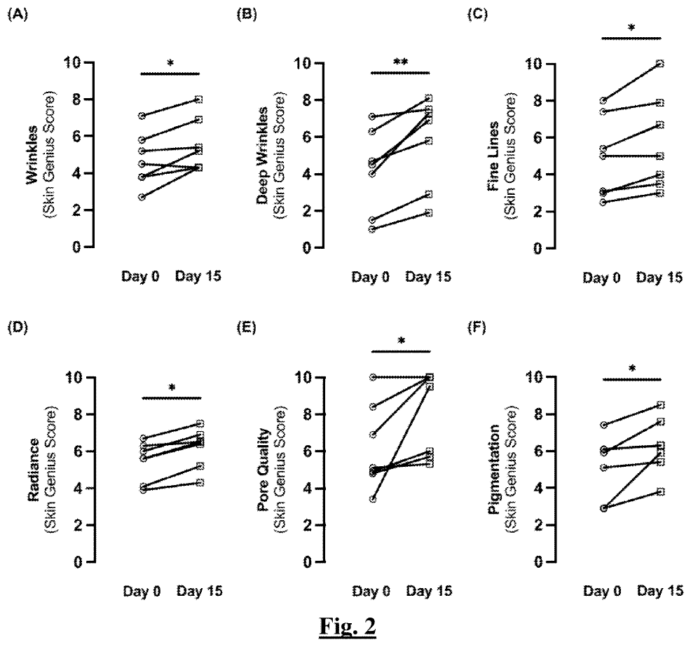

FIG. 2 shows 6 graphs indicating the change of 6 skin attributes after 15 days of topical application of the first embodiment of a cosmetic composition wherein the skin attributes are (A) wrinkles, (B) deep wrinkles, (C) fine lines, (D) radiance, (E) pore quality and (F) pigmentation.

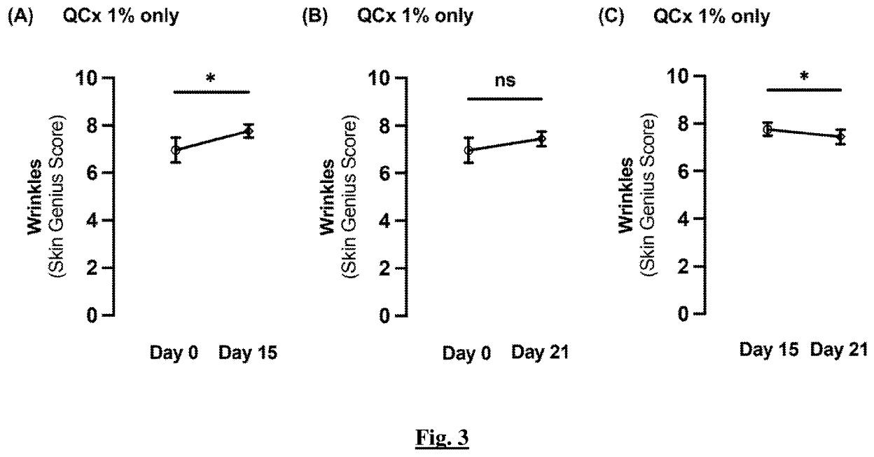

FIG. 3 shows 3 graphs indicating the mean change of the appearance of wrinkles after the 2 weeks of application of a cosmetic composition (composition A) comprising 1 wt. % quercetin and 1 wt. % curcumin (A) and two weeks of applying the composition followed by a 1 week post-trial washout period (day 21) (B) and a graph comparing the appearance of wrinkles at day 15 and day 21 of the trial (C) (error bars indicate mean±SEM).

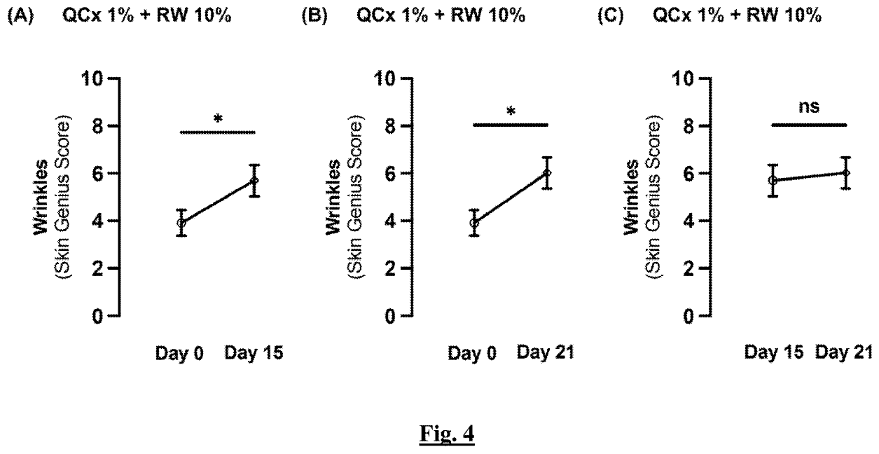

FIG. 4 shows 3 graphs indicating the mean change of the appearance of wrinkles after the 2 weeks of application of a cosmetic composition (composition B) comprising 1 wt. % quercetin, 1 wt. % curcumin and 10 wt. % rose water (hydrosol) (A) and two weeks of applying the composition followed by a 1 week post-trial washout period (day 21) (B) and a graph comparing the appearance of wrinkles at day 15 and day 21 of the trial (C) (error bars indicate mean±SEM).

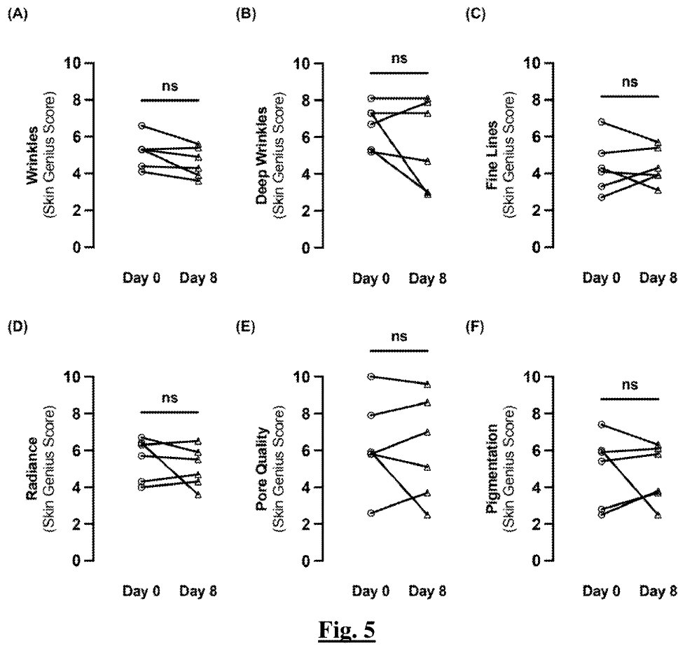

FIG. 5 shows 6 graphs indicating the change of 6 skin attributes after 1 week of topical application of a comparison cosmetic composition wherein the cosmetic composition comprises 0.0013 wt. % quercetin and 0.0013 wt. % curcumin and the skin attributes are (A) wrinkles, (B) deep wrinkles, (C) fine lines, (D) radiance, (E) pore quality and (F) pigmentation.

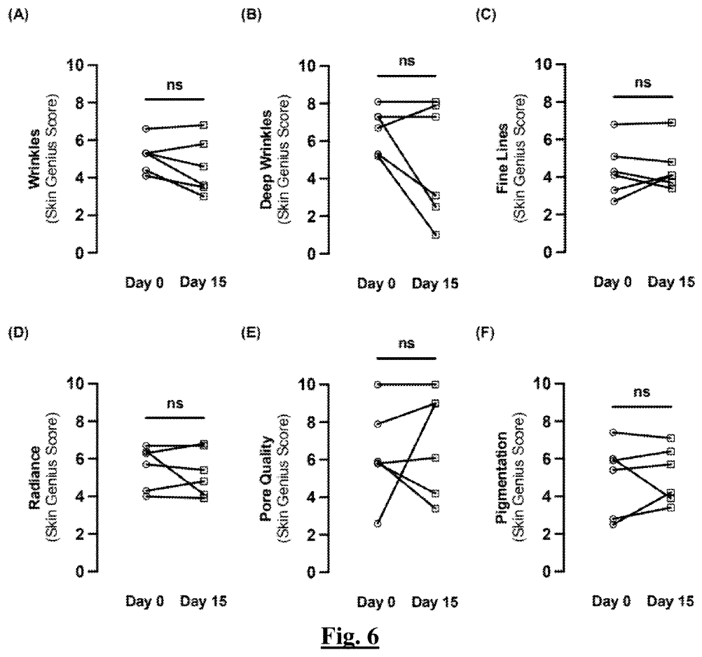

FIG. 6 shows 6 graphs indicating the change of 6 skin attributes after 2 weeks of topical application of a comparison cosmetic composition wherein the cosmetic composition comprises 0.0013 wt. % quercetin and 0.0013 wt. % curcumin and the skin attributes are (A) wrinkles, (B) deep wrinkles, (C) fine lines, (D) radiance, (E) pore quality and (F) pigmentation.

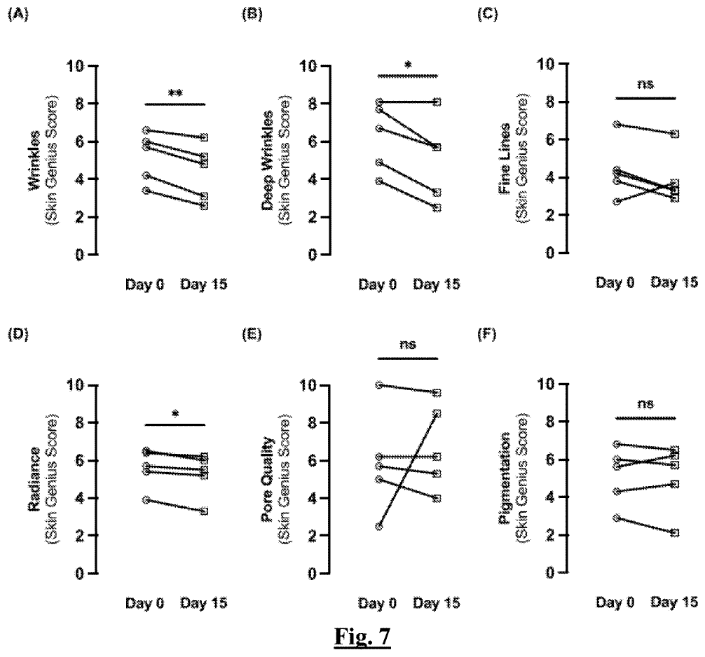

FIG. 7 shows 6 graphs indicating the change of 6 skin attributes after 2 weeks of topical application of a comparison cosmetic composition wherein the cosmetic composition comprises 0.15 wt. % quercetin and 0.15 wt. % curcumin and the skin attributes are (A) wrinkles, (B) deep wrinkles, (C) fine lines, (D) radiance, (E) pore quality and (F) pigmentation.

### EXAMPLE 1

A first embodiment of a cosmetic composition of the first aspect of the invention is provided wherein the cosmetic composition comprises 1 wt. % quercetin, 1 wt. % curcumin, 50 wt. % aloe vera gel, 30 wt. % honey, 10 wt. % rose water (hydrosol), 3 wt. % grapeseed oil and 5 wt. % rice flower.

The cosmetic composition was applied to 7 participants and a number of skin attributes were measured using L'Oreal Skin Genius® analyses (https://www.loreal-paris.co.uk/skin-genius). The attributes were wrinkles, radiance, firmness, pore quality, pigmentation, fine lines and deep wrinkles. The higher the rating, the more improved the attribute for example a higher rated wrinkles attribute means fewer wrinkles were observed.

The participants applied the cosmetic composition to the face twice a day (in the morning and the evening) and a photograph was taken each morning. The results of the rating of the skin attributes are displayed in the graphs of FIG. 1 for day 0 and day 8 and FIG. 2 for day 0 and day 15. The average percentage change in L'Oreal Skin Genius® scores across the attributes of skin aging measured between day 0 and day 15 as it pertains to a first embodiment of a cosmetic composition of the first aspect are shown in table 1.

TABLE 1

| | Average percentage |
|---|---|
| | improvement after 15 days of |
| | application of cosmetic |
| | composition of the invention |
| Skin aging attribute | (skin genius score)/% |
| Wrinkles | +20.04* |
| Radiance | +13.94* |
| Firmness | +13.14^{ns} |
| Pore Quality | +41.22* |
| Pigmentation | +27.23* |
| Fine Lines | +17.44* |
| Deep Wrinkles | +47.63* |

*p < 0.05,
^{ns}= not significant

These results indicated a significant improvement in radiance, pore quality, pigmentation, and fine lines after 1 week of cosmetic composition administration, with a non-significant improvement in wrinkles and deep wrinkles. After 2 weeks of administration, there was a significant improvement in wrinkles and deep wrinkles as well as radiance, pore quality, pigmentation, and fine lines.

### EXAMPLE 2

A second embodiment of a cosmetic composition of the first aspect of the invention (composition A) is provided wherein the cosmetic composition comprises 1 wt. % quercetin, 1 wt. % curcumin, 93 wt. % aloe gel and 5 wt. % rice flower. The cosmetic composition does not comprise 10 wt. % rose water (hydrosol).

A third embodiment of a cosmetic composition of the first aspect of the invention (composition B) is provided wherein the cosmetic composition comprises 1 wt. % quercetin, 1 wt. % curcumin, 10 wt. % rose water (hydrosol), 83 wt. % aloe gel and 5 wt. % rice flower.

Composition A and composition B were tested on participants who applied the cosmetic composition to the face twice a day (in the morning and the evening) for 2 weeks. The appearance of wrinkles was measured after two weeks of application of the compositions (day 15). Following the 2 week trial, participants undertook a 1 week post-trial washout period wherein the participants did not apply either formulation to determine the lasting effects of each of the cosmetic compositions. The appearance of wrinkles was measured after the 1 week post-trial washout period (day 21).

The average percentage change in L'Oreal Skin Genius® scores after two weeks of applying composition A (day 15) and after two weeks of applying composition A and a 1 week post-trial washout period (day 21) and are displayed in the graphs of FIGS. 3A and 3B respectively. FIG. 3C displays a graph showing the comparison between the day 15 and the day 21 data for composition A.

The average percentage change in L′ Oreal Skin Genius® scores after two weeks of applying composition B (day 15) and after two weeks of applying composition B and a 1 week post-trial washout period (day 21) and are displayed in the graphs of FIGS. 4A and 4B respectively. FIG. 4C displays a graph showing the comparison between the day 15 and the day 21 data for composition B.

The data in FIGS. 3A and 4A show that the appearance of wrinkles is significantly improved by each formulation after 2 weeks of twice daily administration.

The data in FIGS. 3B and 4B also show that the appearance of wrinkles is significantly improved by each formulation after 2 weeks of twice daily administration and 1 week of post-trial washout when the data is compared to the day 0 data.

The data in FIGS. 3C and 4C shows that formulation B, which comprises 10 wt. % rose water (hydrosol) in addition to the 1 wt. % curcumin and 1 wt. % quercetin, provides a lasting effect, preventing a decline in the appearance of wrinkles even after the twice daily application has been stopped. It is therefore believed that the inclusion of at least 10% rose water (hydrosol) in the aqueous phase provides an additive and synergistic effect by yielding a persistent cosmetic improvement in the appearance of wrinkles when used in conjunction with curcumin and quercetin, even after the cessation of application, which was not observed to the same extent without rose water (hydrosol). The reduction in the decline in the appearance of wrinkles is advantageous because should the consumer not apply the cosmetic composition for a period of time, they would maintain the improved of skin aging attributes without significant decline.

### EXAMPLE 3

A first embodiment of a topical cosmetic composition of the second aspect of the invention is provided wherein the topical cosmetic composition comprises 1 wt. % quercetin phytosome, 1 wt. % of a mixture of curcuminoids, 50 wt. % aloe vera gel, 30 wt. % honey, 10 wt. % rose water (hydrosol), 3 wt. % grapeseed oil and 5 wt. % rice flower, herein the mixture of curcuminoids comprises 75-80 wt. % of tetrahydrocurcuminoids (THC), 15-20 wt. % of hexahydrocurcuminoids (HHC) and 3-5 wt. % of octahydrocurcuminoids (OHC).

The topical cosmetic composition demonstrated the same performance as the first embodiment of the first aspect of the invention. The first embodiment of the topical cosmetic composition is advantageous because it improves the appearance of skin aging attributes and because it has a high absorption rate.

### EXAMPLE 4

For comparison, two control cosmetic compositions comprising a reduced amount of curcumin and quercetin were prepared. A first control cosmetic composition sample was prepared wherein the control cosmetic composition comprises 0.0013 wt. % quercetin, 0.0013 wt. % curcumin, 51.9974 wt. % aloe vera gel, 30 wt. % honey, 10 wt. % rose water (hydrosol), 3 wt. % grapeseed oil and 5 wt. % rice flower. A second control cosmetic composition sample was prepared wherein the control cosmetic composition comprises 0.15 wt. % quercetin, 0.15 wt. % curcumin, 51.7 wt. % aloe vera gel, 30 wt. % honey, 10 wt. % rose water (hydrosol), 3 wt. % grapeseed oil and 5 wt. % rice flower. The amount of quercetin and curcumin is outside of the range of the invention in both the first control cosmetic composition and the second cosmetic control composition. The control cosmetic composition samples were tested according to the same test method as the inventive cosmetic composition for 2 weeks. The results for the first control sample after 1- and 2-weeks of administration are shown in FIG. 5 and FIG. 6 respectively. The results for the second control sample 2-weeks of administration are shown in FIG. 7.

The average percentage change in L'Oreal Skin Genius® scores across the attributes of skin aging measured between day 0 and day 15 as it pertains to the first control cosmetic composition comprising 0.0013 wt. % quercetin and 0.0013 wt. % curcumin are shown in table 2.

TABLE 2

| | Average percentage |
|---|---|
| | improvement after 2 weeks of |
| | application of cosmetic |
| | composition of the invention |
| Skin aging attribute | (skin genius score)/% |
| Wrinkles | −13.21^{ns} |
| Radiance | −4.02^{ns} |
| Firmness | −9.46^{ns} |
| Pore Quality | +32.55^{ns} |
| Pigmentation | +10.73^{ns} |
| Fine Lines | +6.78^{ns} |
| Deep Wrinkles | −28.35^{ns} |

*p < 0.05,
^{ns}= not significant

The average percentage change in L'Oreal Skin Genius® scores across the attributes of skin aging measured between day 0 and day 15 as it pertains to the second control cosmetic composition comprising 0.15 wt. % quercetin and 0.15 wt. % curcumin are shown in table 3.

TABLE 3

| | Average percentage |
|---|---|
| | improvement after 2 weeks of |
| | application of cosmetic |
| | composition of the invention |
| Skin aging attribute | (skin genius score)/% |
| Wrinkles | −16.98** |
| Radiance | −6.68* |
| Firmness | −4.83^{ns} |
| Pore Quality | +8.77^{ns #} |
| Pigmentation | −3.40^{ns} |
| Fine Lines | −8.09^{ns} |
| Deep Wrinkles | −21.89* |

*p < 0.05,
**p < 0.01,
^{ns}= not significant
^{#}reported as median percentage change owing to outliers

The results indicated no demonstrable significant effect of the cosmetic composition comprising 0.0013 wt. % quercetin and 0.0013 wt. % curcumin on any skin attribute measured after both 1 week and 2 weeks of administration. In particular, the results show that the appearance of wrinkles, deep wrinkles, radiance and firmness of the skin were worse after 2 weeks of administration. This confirms that the cosmetic composition comprising 0.0013 wt. % quercetin and 0.0013 wt. % curcumin is not effective in improving skin aging attributes after 2 weeks of administration.

The results also indicated that the cosmetic composition comprising 0.15 wt. % quercetin and 0.15 wt. % curcumin was not effective in improving any skin aging metrics after 2 weeks of administration. In particular, the results show that the appearance of wrinkles, deep wrinkles, fine lines, radiance, pigmentation and firmness were worse after 2 weeks of administration. This confirms that the cosmetic composition comprising 0.15 wt. % quercetin and 0.15 wt. % curcumin is not effective in improving skin aging attributes after 2 weeks of administration.

The above embodiment is described by way of example only. Many variations are possible without departing from the scope of the invention as defined in the appended claims.

1. A cosmetic composition comprising: (i) at least one curcuminoid; (ii) quercetin provided in a phytosomal complex (quercetin phytosome) with phosphatidylcholine, the amount of quercetin being expressed as quercetin equivalent; and (iii) a cosmetically acceptable carrier, comprising an aqueous phase that includes at least 10 wt. % rose water (hydrosol).

2. A cosmetic composition according to claim 1 wherein the rose water (hydrosol) is steam-distilled Rosa damascena water (hydrosol) comprising water-soluble phenolic constituents present at 10-15 wt. % of the total composition.

3. A cosmetic composition according to claim 1 wherein the phosphatidylcholine is derived from sunflower or soy lecithin.

4. A cosmetic composition according to claim 1 wherein the curcuminoid comprises curcumin.

5. A cosmetic composition according to claim 1 wherein the curcuminoid comprises a hydrogenated mixture comprising between 75 wt. % and 80 wt. % tetrahydrocurcuminoids, and between 15 wt. % and 20 wt. % hexahydrocurcuminoids, and between 3 wt. % and 5 wt. % octahydrocurcuminoids.

6. A cosmetic composition according to claim 1 comprising between 0.5 and 5 wt. % curcuminoid and between 0.5 and 5 wt. % quercetin (as quercetin equivalent in the phytosome).

7. A cosmetic composition according to claim 6 comprising between 0.5 wt. % and 2 wt. % curcuminoid and between 0.5 wt. % and 2 wt. % quercetin (as quercetin equivalent in the phytosome).

8. A cosmetic composition according to claim 7 comprising between 0.5 wt. % and 1.5 wt. % curcuminoid and between 0.5 wt. % and 1.5 wt. % quercetin (as quercetin equivalent in the phytosome).

9. A cosmetic composition according to claim 1 wherein the ratio of the amount of curcuminoid to the amount of quercetin (as quercetin equivalent) is between 1:1 and 1:3.

10. A cosmetic composition according to claim 9 wherein the ratio of the amount of curcuminoid to the amount of quercetin (as quercetin equivalent) is 1:1.

11. A cosmetic composition according to claim 1 wherein the quercetin phytosome is a non-covalent complex of quercetin and phosphatidylcholine prepared by solvent evaporation or anti-solvent precipitation, with a quercetin:phosphatidylcholine molar ratio of 1:1 to 1:3.

12. The composition of claim 1 further comprising one or more of: aloe vera gel (45-55 wt. %), honey (25-35 wt. %), and grapeseed oil (4-8 wt. %).

13. A cosmetic composition comprising: (i) 0.5 wt. % and 1.5 wt. % of at least one curcuminoid wherein the curcuminoid comprises a hydrogenated mixture comprising between 75 wt. % and 80 wt. % tetrahydrocurcuminoids, and between 15 wt. % and 20 wt. % hexahydrocurcuminoids, and between 3 wt. % and 5 wt. % octahydrocurcuminoids; (ii) 0.5 wt. % and 1.5 wt. % of quercetin provided in a phytosomal complex (quercetin phytosome) with phosphatidylcholine, the amount of quercetin being expressed as quercetin equivalent, wherein the phosphatidylcholine is derived from sunflower or soy lecithin and the quercetin phytosome is a non-covalent complex of quercetin and phosphatidylcholine prepared by solvent evaporation or anti-solvent precipitation, with a quercetin:phosphatidylcholine molar ratio of 1:1 to 1:3; and (iii) a cosmetically acceptable carrier, comprising an aqueous phase that includes between 10 wt. % and 15 wt. % rose water (hydrosol) wherein the rose water (hydrosol) is steam-distilled Rosa damascena water (hydrosol) comprising water-soluble phenolic constituents; wherein the ratio of the amount of curcuminoid to the amount of quercetin (as quercetin equivalent) is 1:1.

14. The composition of claim 13 further comprising one or more of: aloe vera gel (45-55 wt. %), honey (25-35 wt. %), and grapeseed oil (4-8 wt. %).

15. A cosmetic composition according to claim 1 wherein, following twice daily topical application of the cosmetic composition for at least 14 days, the appearance of wrinkles remains improved relative to baseline for at least 7 days following cessation of the topical application of the cosmetic composition.

16. A cosmetic composition according to claim 13 wherein, following twice daily topical application of the cosmetic composition for at least 14 days, the appearance of wrinkles remains improved relative to baseline for at least 7 days following cessation of the topical application of the cosmetic composition.

17. A non-therapeutic cosmetic composition according to claim 1 wherein the cosmetic composition is applied to the face (including under the eyes) and/or neck and/or body and/or hair and/or nails and is selected from the group consisting of: a cream, a gel, a balm, a lotion, a mask, a mousse, an oil, a serum, a solution and a spray.

18. A non-therapeutic cosmetic composition according to claim 13 wherein the cosmetic composition is applied to the face (including under the eyes) and/or neck and/or body and/or hair and/or nails and is selected from the group consisting of: a cream, a gel, a balm, a lotion, a mask, a mousse, an oil, a serum, a solution and a spray.

19. Non-therapeutic use of a cosmetic composition according to claim 1 comprising applying a cosmetic composition to cosmetically improve the appearance of skin, hair, nails, and/or at least one skin attribute selected from the group consisting of: wrinkles, deep wrinkles, fine lines, derated radiance, reduced firmness, derated pore quality, increased pigmentation and increased scarring and any combination thereof.

20. Non-therapeutic use of a cosmetic composition according to claim 13 comprising applying a cosmetic composition to cosmetically improve the appearance of skin, hair, nails, and/or at least one skin attribute selected from the group consisting of: wrinkles, deep wrinkles, fine lines, derated radiance, reduced firmness, derated pore quality, increased pigmentation and increased scarring and any combination thereof.
